(19) United States
(12) Patent Application Publication
(10) Pub. No.: US 2025/0017951 A1
(43) Pub. Date: Jan. 16, 2025

(54) ALTERING CANCER CELLULAR FUNCTIONS THROUGH PROTON MOPPING

(71) Applicant: Michael Theodore Lytinas, Roslindale, MA (US)

(72) Inventor: Michael Theodore Lytinas, Roslindale, MA (US)

(21) Appl. No.: 18/425,648

(22) Filed: Jan. 29, 2024

Related U.S. Application Data
(63) Continuation of application No. 17/142,163, filed on Jan. 5, 2021, now abandoned.
Application No. 17/142,163 is a continuation of application No. 15/925,170, filed on Mar. 19, 2018, now abandoned.
Application No. 15/925,170 is a continuation of application No. 14/479,619, filed on Sep. 8, 2014, now Pat. No. 9,919,000.
(60) Provisional application No. 61/960,170, filed on Sep. 12, 2013.

(51) Int. Cl. A61K 31/7028; A61K 31/70; A61K 31/7004; A61K 47/60

(52) U.S. Cl. CPC A61K 31/7028; A61K 47/60; A61K 31/70; A61K 31/7004

(57) ABSTRACT

Proton mopping is a new anti-cancer therapeutic approach that disrupts cancer's immunologic balance. It results in the alteration of the pHi/pHe ratio (intracellular-extracellular) of the cancer cell, leading cancer to either normalcy or apoptosis. This technology deploys a chemical compound that has two parts, a glucose part to guide the molecule to the cancer site and a proton neutralizer to mop up the protons. Proof of the validity of this therapeutic approach came after using an existing chemical compound 2-[2(2-Aminoethoxy)ethoxy]ethyl a-D-mannopyranoside, C₁₂H₂₅NO₈, which has the above mentioned properties. This compound is of the class of functionalized PEGylated glycosides, which are ligands for conjugation to biological molecules.

### BACKGROUND OF THE INVENTION

### Field of the Invention

The present technology describes a new way of treating malignant solid tumors.

### Related Art

Historically, cancer fighting has been about blocking, inhibiting or binding to specific molecular targets. It is either the classic chemotherapeutic approach or the targeted therapy or the immunological therapies. Taxol and 5-FU are both classic examples of the chemotherapeutic approach. Tamoxifen and Herceptin are examples of targeted therapies and Rituxan and Erbitux are examples of immunological therapies with monoclonal antibodies. All of these therapeutic approaches target either directly the cancer cell or rapidly dividing cells in various phases of division. The problem with the above mentioned therapeutic approaches is that they all have the cell or parts of the cell, as a target. However, the immense plasticity of cancer helps cancer to evade being treated as a target, therefore escaping treatment.

### BRIEF SUMMARY OF THE INVENTION

In normalcy, the intracellular space is slightly acidic and the extracellular space is slightly alkaline. In cancer, this dogma is reversed. The intracellular space is slightly alkaline and the extracellular space is slightly or severely acidic. There is a new founded flow of hydrogen ions from the inside of the cancer cell to the outside due to overproduction of protons (H⁺). This technology disrupts said cancer balance and changes the pH inside and outside of the cell. It does so by mopping the excess of protons that are produced by the cancer and guides the cancer cell back to normalcy or apoptosis. Fewer protons means altered pH and altered pH means altered cellular functions. In the case of cancer, it means back to normalcy or apoptosis. This way the suggested technology evades the immense plasticity of the cancer and treats cancer through its microenvironment.

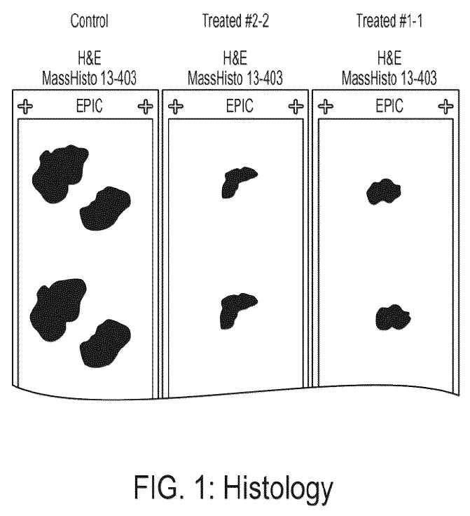

FIG. 1: Histology slides of the experiments show the untreated (control) group on the left and the treated group on the right. Gross size reduction was our goal. The maximal tumor growth inhibition index (T/C ratio) was around 48% (by day 10).

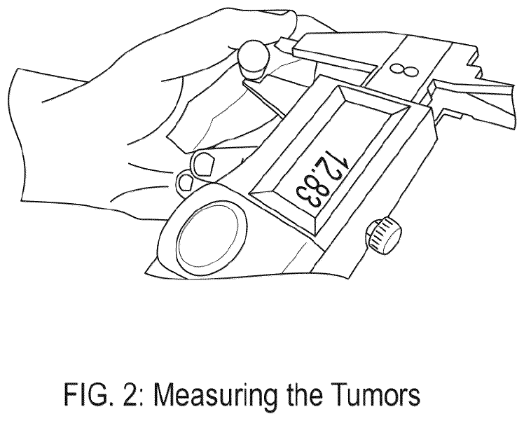

FIG. 2: The gross size reduction of the tumor was measured by using calipers.

### DETAILED DESCRIPTION OF THE INVENTION

### What is the Design of the Chemical Compound?

The chemical compound that is described by the new technology has two parts. The first part is a glucose molecule and the second part is a proton neutralizer. In this case, the proton neutralizer is an amine (NH₂). Said molecule needs two parts because it first needs to reach cancer and then deliver the proton mopping part.

### What is the Synthesis Procedure of this Chemical Compound?

### Synthesis of 1-(2-(2-(2-Aminoethoxy)ethoxy)ethoxy-D-mannopyranoside (5)

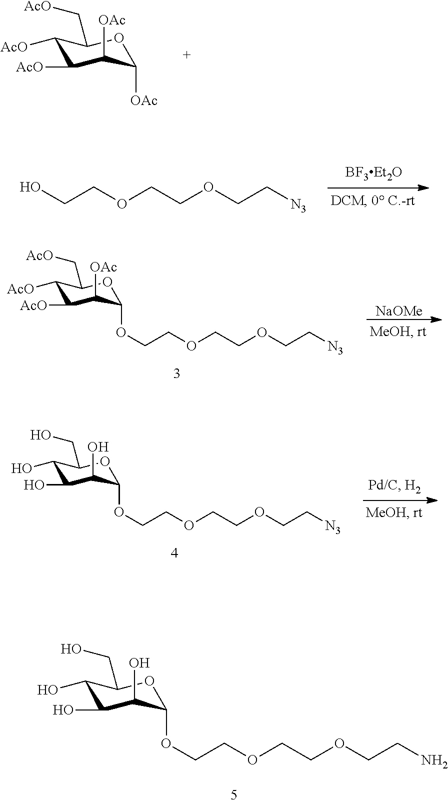

A solution of penta-O-acetate-α-D-mannopyranoside (300 mg, 0.77 mmol) and 2-(2-(2-azidoethoxy)ethoxy)ethanol (200 mg, 1.1 mmol) in dry dichloromethane (5 mL) was cooled to 0° C. BF₃Et₂O (550 mg, 3.8 mmol) was added drop-wise, and the solution was stirred at rt overnight. The solution was poured into ice water, extracted with dichloromethane, and the extracts were washed with saturated NaHCO₃, brine and dried over MgSO₄. The crude product was purified by column chromatography on silica gel using hexane-ethyl acetate (1/1 v/v) as eluent, yielding compound 3 (152 mg, 43%) as a colorless oil. H-NMR (500 MHz, CDCl₃): δ 5.37 (dd, J=3.5 Hz, 1H), 5.28 (d, J=9.5 Hz, 1H), 5.26 (t, J=3.0 Hz, 1H), 4.86 (d, J=1.0 Hz, 1H), 4.28 (dd, J=5.0 Hz, 1H), 4.10 (d, J=2.0 Hz, 1H), 4.07 (m, 1H), 3.81 (m, 1H), 3.67 (m, 9H), 3.39 (t, J=5.0 Hz, 2H), 2.15 (s, 3H), 2.10 (s, 3H), 2.04 (s, 3H), 1.99 (s, 3H). C-NMR (125 MHz, CDCl₃): δ 170.8, 170.1, 170.03, 169.8, 97.8, 70.9, 70.8, 70.2, 70.2, 69.7, 69.2, 68.5, 67.5, 66.3, 62.5, 50.8, 21.0, 20.8, 20.8.

Compound 3 (128 mg, 0.24 mmol) was dissolved in dry methanol (2 mL), and NaOMe (6.8 mg, 0.12 mmol) was added. The reaction mixture was stirred at rt for 1 h. Amberlite IR-120 H⁺ resin was added to adjust the pH to 7. The mixture was then filtered and the solvent was evaporated to yield compound 4 (92 mg, quant) as a colorless oil. H-NMR (500 MHz, D₂O): δ 4.88 (d, J=1.5 Hz, 1H), 3.96 (dd, J=1.5 Hz, 1H), 3.88 (m, 2H), 3.82 (m, 1H), 3.71 (m, 9H), 3.65 (m, 3H), 3.50 (t, J=5.0 Hz, 2H,). C-NMR (125 MHz, D₂O): δ 99.9, 72.7, 70.4, 69.9, 69.6, 69.5, 69.4, 69.2, 66.7, 66.3, 60.9, 50.1.

Compound 4 (82 mg, 0.24 mmol) was dissolved in dry methanol (3 mL), and Pd/C (23 mg) was added. The flask was purged with N₂ and filled with H₂. The mixture was stirred vigorously at rt for 1 h. The reaction mixture was then filtered through Celite and the solvent was evaporated to yield compound 5 (23 mg, 26%) as a clear oil. H-NMR (500 MHz, D₂O): δ 4.88 (s, 1H), 3.95 (t, J=1.5 Hz, 1H), 3.70 (m, 16H), 2.80 (t, J=5.5 Hz, 1H,). C-NMR (125 MHz, D₂O): δ 99.9, 72.7, 70.4, 69.9, 69.6, 69.4, 69.3, 66.7, 66.3, 60.9, 47.4. MS (ESI): [M+H]⁺ calculated for C₁₂H₂₅NO₈: 312.3, found: 312.1.

### Why Do We Need Two Parts in this Chemical Compound?

The first part, the glucose, is needed to guide the compound to the cancer site. It is well known and established in the literature that the majority of solid tumors have an affinity for glucose consumption and they metabolize glucose either aerobically or anaerobically. This glucose part is the guidance system of the chemical compound. Example of a similar situation is the use of FDG to image cancer metastasis in the PET scan. The glucose part of the FDG (Fluoro-Deoxy-Glucose) is guiding the-in this case radioisotope for imaging-to the cancer sites. The second part is the actual proton neutralizer. In this case we use an amine (NH₂). This amine mops up a proton from inside the cancer cell and it becomes NH₃. That is how the disruption of the proton flow occurs and consequently how the pH modification occurs.

### Where Does this Chemical Compound Enter the Cell from?

Said chemical compound enters the cancer cell from the glucose receptors, especially the GLUT-1. Glucose receptors are over expressed in the majority of solid tumors, which facilitates the entrance of the glucose including molecule.

### Which Pathway Does this Chemical Compound Use, Once Inside the Cell?

It uses the hexokinase pathway, the well-known and documented cellular glucose consumption metabolic pathway.

### How Does Proton Mopping Alter Cancer Cellular Functions?

By mopping the excess protons of the cancer's altered metabolism, this chemical compound alters the cancer's pHi/pHe ratio (intracellular-extracellular). It is of paramount importance to cancer to preserve said altered pHi/pHe in order to evade immunologic surveillance and to metastasize to other parts of the human body, as well as gain chemo and radio resistance (chemotherapy-radiotherapy). Cancer is so sensitive to this new pH ratio that even the slightest variation of this ratio, as low as 0.1 pH units or less, may disrupt important biochemical and/or biological processes such as ATP synthesis, enzyme function and the proliferation, migration, invasion and metastasis of tumor cells. Consequently, by altering this pH ratio, cancer is guided back to either normalcy or apoptosis.

### Who Else Talks about pH Manipulation as a Cancer Therapy?

1. The chemistry, physiology and pathology of pH in cancer
   Pawel Swietach, Richard D. Vaughan-Jones, Adrian L. Harris and Alzbeta Hulikova
   Phil. Trans. Royal Society B 2014 369, 20130099, published 3 Feb. 2014
2. Interfering with pH regulation in tumours as a therapeutic strategy
   Dario Neri and Claudiu T. Supuran
   NATURE REVIEWS|DRUG DISCOVERY VOLUME 10| OCTOBER 2011|767
3. Dysregulated pH: a perfect storm for cancer progression
   Bradley A. Webb, Michael Chimenti, Matthew P. Jacobson and Diane L. Barber
   NATURE REVIEWS|CANCER VOLUME 11|SEPTEMBER 2011|671
4. Cellular pH gradient in tumor versus normal tissue: potential exploitation for the treatment of cancer
   Leo E. Gerweck and Kala Seetharaman
   CANCER RESEARCH 56, 1194-1198 Mar. 15, 1996
5. DNA Damage-Induced Bcl-xL Deamidation Is Mediated by NHE-1 Antiport Regulated Intracellular pH
   Rui Zhao, David Oxley, Trevor S. Smith, George A. Follows, Anthony R. Green, Denis R. Alexander
   PLOS Biology|www.plosbiology.org 0039 January 2007|Volume 5|Issue 1|e1
6. Influence of Tumor pH on Therapeutic Response
   Chang W. Song, PhD, Robert Griffin, PhD, and Heon Joo Park, MD, PhD
   From: Cancer Drug Discovery and Development: Cancer Drug Resistance
   Edited by: B. Teicher© Humana Press Inc., Totowa, NJ

### Who Else Uses PEGylated Glycosides to Deliver Therapeutic Molecules?

1. U.S. Pat. No. 8,063,015 B2,
2. US 2003/0031671 A1

Both patents and their references are about using several PEGylated glycosides as a vehicle to deliver either monoclonal antibodies or other therapeutic molecules to cancer.

### How Does this Technology Differ from Other Cancer Therapies?

The suggested technology does not target the cancer cell per se like all other technologies do. The immense plasticity of the cancer cells makes it very hard for other therapeutic approaches to guide cancer to a different state. The suggested technology deals with the micro environment that cancer creates to favor its survival. This micro environment is the altered pH ratio from inside and outside of the cell. By disrupting this micro environment, which is manifested by the cancer's new pHi/pHe ratio, the new technology evades cancer's immense plasticity by not directly dealing with the cancer cells themselves. In contrast, all the existing technologies target cancer cells in an attempt to kill them. In the classic chemotherapeutic approach, the target is all rapidly dividing cells, which includes normal cells, hence the tremendous side effects.

### How Do We Know that this Technology with Said Compound Works?

There are preliminary clinical results in mice that prove that the compound of the suggested technology works on a syngeneic mouse breast cancer model.

[FIG. 1] This is the human breast cancer analog, which shows 48% tumor reduction.

(The protocol of the experiment follows in the next page)

### Protocol of the Experiment

The Purpose: to assess the anti-tumor activity of the existing compound, C₁₂H₂₅NO₈, PEGylated amine functionalized glycoside, 2-[2(2-Aminoethoxy)ethoxy]ethyl a-D-mannopyranoside, on a model of mouse 4T1 breast tumor in Balb/C mice.

Animal and Facility: healthy female Balb/c (5-6 weeks old), purchased from Harlan Laboratories, were allowed to acclimate for 3 days in a specific pathogen free animal facility. Animal housing, handling and procedures were followed according to the protocols and guidelines approved by the lab's Institutional Animal Care and Use Committee (IACUC).

In Vivo Syngeneic Tumor Study Design: Murine 4T1 breast tumor cells were kept in RPMI-1640 medium containing 10% FBS and 1% Pen/Strep. On the day of inoculation, the cells were harvested according to standard protocol (Beth Pulaski) and re-suspended in PBS at the cell concentration of 2.5×105 cells/ml. Each animal was injected with 0.1 ml of the cell solution into the right flank of Balb/c mice following the SOP. On the 8th day post-inoculation, all tumor volumes were measured and the animals were divided into two groups, control and treated.

Study Design: to deliver a predetermined dose of the compound intravenously for 5 days.

Study Objective: to observe clinically the reduction of the size of the tumors using calipers (our “biomarker”). [FIG. 2]

Treatment: the tested compound was prepared right before dosing on day 8 after 4T1 inoculation. Each mouse received a predetermined dose of either the compound or saline intravenously daily for 5 consecutive days. The mice tumor volumes and mice body weights were monitored every other day along with other general behavior factors.

Endpoints: animal was euthanized if one of the following conditions was identified:
its neoplasm reached the predetermined 1000 cubic mms
the animal became moribund
body weight lost more than 20% of its original weight

Results: In the study, the 4T1 tumor growth was similar to our previous data. The control tumors were doubled every 2-3 days. However, the tumors in the treated group with the compound grew slowly. This means that the compound can inhibit 4T1 tumor growth during treatment (from day 8 to day 12). In fact, the maximal tumor growth inhibition index (T/C ratio) was around 48% (by day 10) [FIG. 1]. The inhibitory effect on the tumor growth by the compound was tolerated by the mice and no toxic effects were observed.

1. A cancer therapeutic method comprising administering a proton (hydrogen ion) mopping compound to a subject.

2. A proton mopping compound for use in cancer therapy.

3. The compound of claim 2, wherein said compound has two parts, one for targeting cancer cells and one for mopping up the protons.

4. The compound of claim 2, comprising a targeting part that is a member of the glucose family or equivalent or analog to glucose.

5. The compound of claim 2 further comprising a proton mopping part that is a member of the amine NH2 family or equivalent proton acceptor.

6. The compound of claim 2, which enters cancer cells and mops up excess protons (hydrogen ions) produced by cancer metabolism.

7. The compound of claim 2, which alters the pH ratio between the intracellular and the extracellular space of cancer cells.

8. The compound of claim 2, which alters cancer metabolism by altering cancer cell pHi/pHe (intra-extracellular), thereby guiding cancer cells to either normalcy or apoptosis.

9. The method of claim 1, wherein said compound has molecular formula C12H2sNOs and name 2-[2(2-Aminoethoxy)ethoxy]ethyl a-D-mannopyranoside.
